(19) United States
(12) Patent Application Publication
(10) Pub. No.: US 2007/0299409 A1
(43) Pub. Date: Dec. 27, 2007

(54) Antimicrobial Needle Coating For Extended Infusion

(75) Inventors: Richard J. Whitbourne, Rochester, NY (US); Margaret Lydon, North Chili, NY (US); David M. Gravett, Vancouver (CA)

Correspondence Address: VENABLE LLP, P.O. BOX 34385, WASHINGTON, DC, 20043-9998, US

(73) Assignee: Angiotech Biocoatings Corp., Henrietta, NY (US)

(21) Appl. No.: 11/667,311

(22) PCT Filed: Nov. 9, 2005
(86) PCT No.: PCT/US05/40512

§ 371 Date: May 9, 2007

Related U.S. Application Data
(60) Provisional application No. 60/625,958, filed on Nov. 9, 2004.

(51) Int. Cl. A61M 5/00

(52) U.S. Cl. 604/264; 600/365

(57) ABSTRACT

The present invention relates to bioabsorbable polymeric coatings with antimicrobial agents that provide coated surfaces that resist protein absorption and infectious formation on coated surfaces of medical devices that are inserted or implanted in patients, and kits thereof with an antimicrobial disc.

### FIELD OF THE INVENTION

The present invention relates to coated insertable or implantable medical devices having anti-infective, anti-protein absorption properties capable of reducing the incidence and/or severity of infections occurring at or associated with the site of insertion or implantation on the bodily surface of such devices, and extending the patency of the device after insertion or implantation.

### BACKGROUND

In the course of placing an insertable or implantable device in a patient, contamination can occur, as individuals often proceed in an ad hoc fashion. Typically, when a needle or catheter is inserted, the area of insertion is cleaned with an antiseptic. For example, wiping the area with a swab saturated with 70% alcohol can accomplish this. Often, the site will be palpated after swabbing, occasionally contaminating the site.

When such devices are left in place, even for a few days, infections often result. Exudate often seeps from the insertion site. The exudate picks up skin flora which can diffuse back into the patient along the wetted device surface, thereby causing infection.

Another consequence of inserting a medical device such as a sensor or a needle for use in administering medicaments or nutrients is that a cascade of absorption of proteinacous material begins on the device surface. The absorbed protein encapsulates the implanted device with a layer that gradually increases in thickness as the absorption process continues. Within three to five days, the absorbed protein layer is of such magnitude that it may interfere with the detection properties of a sensor, or absorption of medicaments and/or nutrients that are being administered through the inserted medical device. In cases such as insulin pump needles, the protein encapsulation process, together with risk of infection, make it necessary to exchange the inserted needle at two to three day intervals. The need for such frequent exchanges of inserted devices is not convenient, and poses greater risks of inserting a device that may have been inadvertently contaminated with infectious organisms.

Many investigators have tried to solve these problems. For example, placement of a junction seal after placement of a urinary catheter has been examined for preventing bacteriuria and reducing mortality. In one study, the incidence of bacteriuria was higher in a control group than in a treatment group for some of the potential risk factors studied; yet the differences were not statistically significant. (T. S. Huth, Arch. Intern. Med. 152:807, 1992). Lubricants containing polymyxin B or placebo were used with catheters impregnated with tetramethylthiuramdisulfide and a cyclic thiohydroxamic proprietary agent and no significant difference between these types of catheters and catheter care was seen (H. K. I. Bulter, J. Urol. 100:560, 1968). Catheters designed for instillation of intraurethral antibacterial lubricants also were not efficacious in reducing the incidence of infections. (C. M. Kunin, J. Urol. 106:928, 1971). Further, initial reports that silver coating of catheters prevented the adherence and growth of Escherichia coli and Pseudomonas aeroginosa in vitro without causing cell toxicity, led to the use of silver oxide urinary catheters. (H. Liedberg, J. Urol. 17:357, 1989; H Liedberg, Urol. Res. 17:359, 1989). However, a large clinical trial of silver oxide coated urinary catheters in selected patients yielded similar rates of infection between the silver coated group and the uncoated control silicone catheter group. (J. R. Johnson, J. Infect. Dis. 162:1145, 1990). Coated central venous catheters demonstrated a lower catheter colonization rate than observed in uncoated controls. (Veentra, JAMA 281:261, 1999; Collins, Chest 115:1632, 1999). In contrast, other studies reported no benefit to the use of coated central venous catheters. (Bach, Crit. Care Med. 27:515 1999).

In a different field, some wound healing products contain films or hydrogel layers, which may be wetted with liquid materials to promote wound healing. For example, hydrogel wound dressing products are described in U.S. Pat. Nos. 5,204,110, and 5,112,618. Examples of bandages for wound dressings that contain therapeutic agents are described in U.S. Pat. Nos. 5,260,066 and 5,322,695. However, such products are not suitable for limiting infection at the insertion site of an insertable or implantable medical device, nor infections and protein absorption on the surface of an implanted medical device.

Biomimetic hydrogels containing acrylamide-functionalized carbohydrate, sulfoxide, sulfide or sulfones copolymerized with a hydrophilic or hydrophobic material are disclosed in U.S. Pat. No. 6,552,103, providing some protection against protein absorption, but only for testing periods up to 72 hours, not up to seven to ten days. An infusion cannula is provided in U.S. Pat. No. 6,475,196 that is prepared with a polymer coating that contains antimicrobial agents. U.S. Pat. No. 6,368,611, discloses devices having anti-infective coatings and U.S. Pat. No. 6,340,465, reaches the use of lubricious coatings for medical devices. However, these references do not address the issue of protein absorption and long patency. Medical devices containing polyarylate random block copolymers with poly (alkylene oxide) are provided in U.S. Pat. No. 6,319,492. These coatings may show activity against adhesions between injured tissues, but do not address protein encapsulation and infection.

Hence, there remains a need for methods and products for limiting the degree of contamination, including preventing or reducing the growth of microorganisms within an exudate, at the insertion site of an insertable or implantable medical device, reducing or preventing protein absorption and infectious growth on the surface of an inserted or implanted medical device such as a sensor or an insulin pump cannula, and further increasing the patency of inserted or implanted devices.

### SUMMARY OF THE INVENTION

The present invention relates to insertable or implantable devices with surfaces comprising anti-protein absorption agents, such as bioabsorbable polymers, and bioactive agents, such as antimicrobial agents, that provide surfaces that extend the patency of the devices, e.g., by resisting or reducing both protein absorption and infectious formation on surfaces of medical devices that are inserted or implanted in patients.

The present invention relates to an insertable or implantable medical device comprising a percutaneously insertable surface, which comprises a surface layer that comprises at least one anti-infective agent and at least one polymer that is effective to substantially extend and impart extended patency of the device when inserted into a patient. In one aspect of the invention, the surface layer is coated with a coating composition, solution or formulation comprising at least one anti-infective agent and at least one anti-protein absorption bioerodable polymer.

In an exemplary embodiment, the surface layer may be deciduous. The device may be a needle. The device may be one that is inserted into a subject, a portion of the device protruding out of the subject, or inserted into tissue, a portion of the device protruding out of the tissue. In an exemplary aspect, the device may be an implantable medical device, wholly implanted inside a subject.

The insertable medical device may be a needle, an infusion set or device, a peripheral venous catheter or needle, an indwelling infusion needle, a butterfly needle, a subcutaneous access device, an insulin pump needle, a patient controlled analgesia (PCA) pump needle, an arterial catheter, a central venous catheter, a dialysis catheter, a peritoneal dialysis catheter, a nephrostomy catheter, a percutaneous cystostomy catheter, an indwelling paracentesis or pleurocentesis catheter or drain, a percutaneous nephrostomy, a cystostomy tube, a spinal or epidural catheter or a sensor.

In one aspect of the invention, the surface layer may be on less than the entire inserted portion of the device, the entire inserted portion of the device or the entire device. Examples of a device include an intradermal needle, an insulin pump needle or a blood glucose monitor. In certain aspects, about 1.5 cm of the needle is coated and about 1.0 to about 1.5 cm of the needle is inserted into the subject.

In an exemplary embodiment, the polymer may be biocompatible and bioabsorbable and the device surface layer resists or reduces protein encapsulation. In one aspect, the bioerodable polymer comprises a water soluble polymer or a dispersible polymer. Examples of bioerodable polymers include polyethylene glycol, polyethylene oxide, acrylic acid or a salt or a copolymer thereof, acrylic emulsion copolymer, a polymer or copolymer of lactic acid, a polymer or copolymer of glycolic acid, polyacrylamide, polyvinylpyrrolidone, polyurethane, and water-soluble cellulose polymer or methylcellulose. Other examples of bioerodable polymers include copolymers of polyethylene glycol or polyethylene oxide, cellulose acetate phthalate, or polyvinylalcohol.

The surface layer may comprise about 50% to about 99.9% or about 70% to about 99% bioerodable polymer. In another aspect, the bioerodable polymer may be a higher molecular weight polyethylene glycol (PEG), e.g., having a molecular weight of at least about 3500. The polyethylene glycol (PEG) may have a molecular weight of at least about 3500 to 35,000, i.e., PEG 3500, PEG 8000, PEG 10,000, PEG 20,000, PEG 30,000 or PEG 35,000. The bioerodable polymer may comprise PEG 8000 or PEG 20000. The bioerodable polymer may be MePEG-PDLLA 60:40 or higher molecular weight polyethylene glycol. In another aspect, the surface layer further may comprise acrylic emulsion copolymer, polyethylene-co-acrylic acid polymer, epoxy resin, polyurethane resin or melamine-formaldehyde resin.

The surface layer may further comprise one or more non-bioabsorbable polymers, such as acrylates, urethanes, polycarbonates, polyamides, polyesters and polyimides, styrene isobutylene, styrene polymers, cellulose esters, polystyrene or alkylated polyvinylpyrrolidone. The surface layer may further comprise one or more biostable polymers selected from cellulose ester polymers and copolymers, polyurethanes, polyvinyl chloride, polyamides, acrylate polymers and copolymers, ethylenevinylacetate copolymers, vinylpyrrolidoneethylacetate copolymers, acetal polymers and copolymers, silicone polymers and copolymers, polyesters, polyimides and copolymers or polyetherimides. In one aspect, the surface layer or under layers may comprise at least about 1 to 50% nitrocellulose.

The anti-infective agent may be a quaternary compound, a phenolic compound, an iodinated compound, a silver compound or an acidic-anionic compound. In another aspect, the anti-infective agent may be 2-bromo-2-nitropropane-1,3-diol (BRONOPOL), Irgasan (TRICLOSAN), polyhexamethylene biguanide (BAQUACIL), benzalkonium chloride, benzethonium chloride, cetylpyradinium chloride, stearalkonium chloride, phenol, cresol, aminophenol, iodine, iodide, 8-hydrixyquinolone or chlorhexidine. In another aspect, the anti-infective agent may be 5-fluorouracil or methotrexate.

The surface layer may comprise from about 0.1% to 50%, from about 0.5% to 30% or from about 3% to 27% of one or more anti-infective agents. In another aspect, the surface layer comprises one or more of an anti-infective agent such as benzalkonium chloride, 2-bromo-2-nitropropane-1,3-diol (BRONOPOL), Irgasan (TRICLOSAN), and/or polyhexamethylene biguanide (BAQUACIL). In yet another aspect, the surface layer may comprise 2-bromo-2-nitropropane-1,3-diol (BRONOPOL) and/or polyhexamethylene biguanide (BAQUACIL).

The surface layer may comprise a therapeutic agent, e.g., bactericides, antibiotics, antivirals, antiseptics, antineoplastics, anticancer compounds, antifungals, and/or anti-yeast and anti-scarring agents, such as paclitaxel or an analog or derivative thereof, or rapamycin or an analog or derivative thereof. In another aspect, the surface layer may comprise one or more of bactericides, antibiotics, antivirals, antiseptics, antineoplastics, anticancer compounds, antifungals, and/or anti-yeast and anti-scarring agents, e.g., in an amount of from about 0.01 to 8.0% or from about 0.5 to 5.5%.

In yet another exemplary embodiment, the surface layer may further comprise a corticosteroid, which can be either synthetic or natural, such as dexamethasone, alclometasone dipropionate, amcinonide, betamethasone, clobetasol proprionate, clocortolone pivalate, cortisone, hydrocortisone, desonide, desoximetasone, diflorasone diacetate, fluocinolone acetonide, fluocinonide, fluandrenolide, halcinonide, methylprednisolone, mometasone furoate, or triamcinolone.

In another embodiment, the surface layer may further comprise a non-steroidal anti-inflammatory drug (NSAID), such as aspirin, phenylbutazone, indomethacin, sulindac, tolmetin, ibuprofen, piroxicam, fenamate, acetaminophen, or phenacetin.

In yet another exemplary aspect of the invention, the surface layer may comprise two or more coating layers, which can be a primer, a basecoat and/or a topcoat layer. In one aspect, the primer layer comprises polyethylene-co-acrylic acid polymer, epoxy resin and/or polyurethane resin, the basecoat layer comprises at least one bioerodable and/or at least one biostable polymer, and the topcoat layer comprises an anti-infective agent and/or a bioerodable polymer.

In an exemplary embodiment, the present invention relates to a coating composition (pre-coating solution or formulation) comprising at least one anti-infective agent and at least one bioerodable polymer, wherein the composition, when applied to a percutaneously insertable surface of an insertable or implantable medical device, provides a surface layer that substantially extends the patency of the device when inserted into a patient. The coating composition may comprise from about 0.1% to about 25% or about 5% to about 20% bioerodable polymer and from about 0.01% to 8.0%, about 0.5% to 5.5% or about 0.5% of one or more anti-infective agents.

In one aspect, the composition comprises a solvent such as water, acetonitrile, methylethyl ketone, denatured ethanol, ethanol, saline solution, normal saline solution, tetrahydrofuran, isopropyl alcohol, other alcohols, amines, amides, 1,3-dioxalane, ketones, esters, cyclic compounds, glycols, carboxylic acids and/or aromatic solvents and combinations. The composition may comprise from about 50% to about 99% or from about 90% to about 98% solvent.

In another aspect, the invention may comprise a primer composition, a basecoat composition or a topcoat composition. For example, the primer composition may comprise at least one solvent and at least one biostable polymer or resin, the basecoat composition may comprise at least one solvent and at least one bioerodable and at least one biostable polymer or resin, and the topcoat may comprise at least one solvent, at least one anti-infective agent and at least one bioerodable polymer. In an exemplary embodiment, the primer, basecoat and/or the topcoat composition comprises about 50 to 90% solvent and about 8 to 30% polymer. In one aspect, a primer or basecoat composition comprises nitrocellulose in ethanol, tetrahydrofuran, and benzyl alcohol in a ratio of 2:15:1 by weight.

In another exemplary embodiment, the solvent may be acetonitrile, denatured ethanol, methylethyl ketone, toluene, benzyl alcohol, tetrahydrofuran (THF), cyclohexanone, dibutylphthalate, butanol, xylene, water, isopropyl alcohol, ethanol or ethylbenzene. In one aspect, the primer composition may comprise a polymer such as 5% polyethylene-co-acrylic acid polymer, 37.5% w/w Epoxy resin in THF and/or polyurethane resin 25% in DMA. The basecoat composition may comprise a polymer such as nitrocellulose, polyethylene glycol, melamine-formaldehyde resin, acrylic polymer, and/or polyurethane resin. The topcoat composition may comprise a bioerodable polymer such as MePEG/PDLLA 60/40 and/or polyethylene glycol.

The present invention also relates to a kit for reducing protein absorption and development of infections arising from insertion of a medical device through a body surface comprising: an insertable medical device having a percutaneously insertable surface, means for providing the insertable surface with a patency-extending coating, wherein the coating comprises at least one anti-infective agent and at least one polymer; and a disc comprising at least one anti-infective agent, said disc being adapted to surround and abut said percutaneously insertable surface when the device is inserted in a subject and a portion of said percutaneously insertable surface projects from an external bodily surface of the subject, and said disc is in contact with said external bodily surface of the subject. The device and the disc may be packaged together.

In one aspect, the coating may be formed on the needle. In another aspect, the means for providing the coating comprises a swab or an absorbent pad having a composition comprising at least one anti-infective agent. The device, the disc, and the swab or the absorbent pad may be packaged together or are packaged separately. In yet another embodiment, the disc, the swab, and/or the absorbent pad are saturated with a composition comprising at least one anti-infective agent.

In another exemplary embodiment, the device may be a needle, an infusion set or device, a peripheral venous catheter or needle, an indwelling infusion needle, a butterfly needle, a subcutaneous access device, an insulin pump, a patient controlled analgesia (PCA) pump, an arterial catheter, a central venous catheter, a dialysis catheter, a peritoneal dialysis catheter, a nephrostomy catheter, a percutaneous cystostomy catheter, an indwelling paracentesis or pleurocentesis catheter or drain, a percutaneous nephrostomy, a cystostomy tube, a spinal or epidural catheter, or a sensor.

In an exemplary embodiment less than the entire surface of the device may be coated. The device may be uncoated and the swab may be wetted with a composition comprising at least one anti-infective and at least one anti-protein absorption agent, for coating the surface of the device. The disc may be capable of being penetrated by the device and it may comprise an aperture to accommodate passage of the device. The disc may be placed around the device post insertion. In another aspect, the disc may comprise a multitude of fine perforations and is flexible, inert, porous, a fabric, and/or absorbent. In yet another embodiment, the disc may comprise an absorbent material or it may comprise a non-absorbent material. In another aspect, the disc may comprise material such as foams, films, or woven and non-woven materials, in the form of a gauze, a mesh, or a porous filter material. In another embodiment, the disc may comprise material formed from a polymer such as polyester, polypropylene, and/or polyethylene. In another embodiment, the disc may comprise material such as cotton, cellulose, and/or rayon.

In another exemplary embodiment, the disc may comprise more than one layer. In one aspect, the disc may comprise a first layer for contacting the body surface and permeable to anti-infective, anti-protein absorption agents, and a second layer containing a composition of at least one anti-infective agent in a solvated or dry form. In another aspect, the disc may have an adhesive means for adhering to the body surface. The absorbent pad may be attached to the disc. In another aspect, the absorbent pad may be composed of a material capable of absorbing or being soaked or wetted by the composition comprising at least one anti-infective or anti-protein absorption agent. The absorbent pad may comprise material such as plastic foams, cotton gauzes, and/or porous filter material. In another aspect, one or more components of the kit may be sterile.

In yet another exemplary embodiment, the invention relates to a method of coating an insertable medical device, comprising applying a coating comprising at least one anti-infective agent and at least one polymer, by (a) applying the coating prior to packaging the device and/or (b) coating the device with a moistened swab or pad after removing the device from its package prior to insertion. In one aspect, the coating may be applied by spraying, dipping or wiping. In another exemplary embodiment, the coating may be manufactured using an extrusion process. The coating may be dried at an elevated temperature.

The present invention relates to a method of extending the patency of an untreated insertable medical device comprising treating a surface of the device with a composition comprising at least one anti-infective agent and at least one polymer. The composition may be coated onto the insertable medical device. In one aspect, the composition may reduce the incidence and/or severity of protein absorption and build up on the inserted device or the incidence and/or severity of infections occurring at or associated with the site of insertion of the device. In certain aspects, the device may be inserted and remains patent for at least about 5 days. In another aspect, the device, when inserted, may remain patent for at least about 20% longer than the untreated device.

In another exemplary embodiment, the invention relates to a method of using an insertable medical device, comprising: (a) providing an insertable medical device that has been coated with a composition comprising at least one anti-infective agent and at least one polymer; and (b) inserting the device into a subject. In one aspect, the invention further comprises wiping the surface of the device with a swab or pad having a solution comprising at least one anti-infective agent and at least one polymer, prior to insertion.

In another embodiment, the invention relates to a method for reducing protein absorption and development of infections arising from insertion of a medical device through a body surface comprising coating the device with a composition comprising at least one anti-infective agent and at least one polymer. The invention may be a device which is inserted through a disc comprising an antimicrobial agent. The invention may also comprise placing around the device at the site of penetration a disc comprising an antimicrobial agent.

### BRIEF DESCRIPTION OF THE DRAWINGS

FIG. 1 is a side view of an embodiment of a disc and needle inserted into a patient.

FIG. 2 is a side view of a second embodiment of an inserted disc and needle.

### DETAILED DESCRIPTION

The present invention relates to insertable or implantable devices with surfaces comprising patency-extending, e.g., anti-protein absorption agents, such as bioerodable or bioabsorbable polymers, and bioactive agents, such as antimicrobial/anti-infective agents, that provide surfaces that for example, resist both protein absorption and infectious formation on surfaces effective to substantially extend patency of the medical devices when inserted or implanted in patients.

In an exemplary embodiment, the present invention relates to bioerodable polymeric surface layers with antimicrobial agents that provide coated surfaces that resist or reduce both protein absorption and infectious formation on surfaces of medical devices that are inserted or implanted in patients. Such surfaces are useful on devices that are inserted or implanted in patients for extended periods of time, and which enable such inserted or implanted devices to remain patent substantially longer than devices without such a surface.

“Inserted” refers to a device for which at least a portion has been introduced into a host. A device such as an implant may be inserted into body tissue, for example, through the skin (percutaneously), or other types of tissue, such as muscle, bone, cartilage, tendons, fascia, and the like, or into a body lumen (e.g., a blood vessel) or cavity. A device is partially inserted when some of the device reaches, or extends to the outside of, a host.

“Implanted” refers to an implant device that is placed completely (i.e., the whole implant resides within the host) or partially within a host. An implant or other device is partially implanted when some of the device reaches, protrudes, or extends to the outside of, a host. The terms “insertable device” and “implantable device” are used somewhat interchangeably.

“Host”, “person”, “subject”, “patient”, “individual” and the like are used synonymously to refer to the living being into which a device or implant of the present invention is inserted or implanted. The host may be a human or non-human animal.

In an exemplary embodiment, the invention relates to an insertable medical device having a percutaneously insertable surface, the insertable surface having a surface layer, wherein the surface layer comprises at least one anti-infective agent and at least one anti-protein absorption agent.

Without limiting the scope of the invention, insertable or implantable devices may include devices inserted into tissue, e.g., needles, or devices inserted into vessels or cavities, e.g., catheters. Examples of needles are an infusion set or device, a peripheral venous needle, an indwelling infusion needle, a butterfly needle, a subcutaneous access device, an insulin pump needle or a patient controlled analgesia (PCA) pump needle. Examples of catheters are a peripheral venous catheter, an arterial catheter, a central venous catheter (CVC), a dialysis catheter, a peritoneal dialysis catheter, a nephrostomy catheter, a percutaneous cystostomy catheter, an indwelling paracentesis or pleurocentesis catheter or drain, a percutaneous nephrostomy, a cystostomy tube, a spinal or epidural catheter. Such devices may be used, for example, to introduce various materials such as nutrients or therapeutic agents into patients, or to drain material from a patient. Devices that are not intended for infusion purposes, such as sensors, may also be used.

The devices of the invention may be those inserted into tissue, such as needles, or those inserted into vessels and cavities, such as catheters, a portion of which is inserted into the body of the patient and a portion of which protrudes outside of the body. In another exemplary embodiment, the device may be wholly implanted inside of the body of the patient, e.g., completely beneath the skin surface, such as implantable medical devices. These include, e.g., implantable glucose monitoring devices or implantable insulin pumps. Additional examples of implantable devices may include catheters (e.g., vascular and dialysis catheters), stents, heart valves, cardiac pacemakers, implantable cardioverter defibrillators, grafts (e.g., vascular grafts), ear, nose, or throat implants, urological implants, endotracheal or tracheostomy tubes, CNS shunts, orthopedic implants, and ocular implants. Accordingly, examples include catheters, e.g., central venous (CVC's), hemodialysis and urinary; pacemaker leads, e.g., silicone and polyurethane; tubes, e.g., gastroenteric, drain, nasogastric and endotracheal; shunts, e.g., arteriovenous and hydrocephalous; and needles, e.g. insulin pump, fluid administration, amniocenteses and biopsy. Exemplary embodiments may be devices used to introduce drugs, e.g., insulin using an insulin pump needle, or devices for fluid drainage, e.g., central nervous catheter containing an anti-infective drug, e.g., 5-fluorouracil and/or methotrexate.

In another aspect, a device may include a plurality of reservoirs within its structure, each reservoir configured to house and protect the anti-infective agent. The reservoirs may be formed from divots in the device surface or micropores or channels in the device body. In one aspect, the reservoirs are formed from voids in the structure of the device. The reservoirs may house a single type of drug or more than one type of drug. The drug(s) may be formulated with a polymer (e.g., an anti-protein absorption, bioerodable polymer), which is loaded into the reservoirs. The filled reservoir can function as a drug delivery depot, which can release drug over a period of time dependent on the release kinetics of the drug from the polymer. In certain embodiments, the reservoir may be loaded with a plurality of layers. Each layer may include a different drug having a particular amount (dose) of drug, and each layer may have a different composition to further tailor the amount of drug that is released from the substrate. The multi-layered carrier may further include a barrier layer that prevents release of the drug(s) or modulates the drug release rates. The barrier layer can be used, for example, to control the direction that the drug elutes from the void.

The surface layers of the present invention may be formed using various techniques and methods, for example, wherein at least one anti-infective agent and at least one anti-protein absorption agent, e.g., bioerodable polymer, are used in forming a surface which may be provided in a solution, formulation or composition (pre-coating) which is used to coat the device, or the device may be a plastic needle or catheter with a polymeric surface or the device may be made by extrusion of polymers.

For example, the device may be an insertable or implantable needle or a catheter having a percutaneously insertable surface, the insertable surface having a coating, which comprises at least one anti-infective agent and at least one anti-protein absorption bioerodable polymer. In another aspect of the invention the patency-extending surface is on less than the entire inserted portion of the device surface, the entire surface of the inserted portion or the entire surface of the device.

The invention may relate to a device having a surface layer, e.g., a coating composition, comprising a biocompatible bioerodable/bioabsorbable polymer, wherein the surface layer prevents, reduces or resists protein encapsulation of the inserted or implanted device. The surface layer may comprise anti-infection and anti-protein absorption agents, e.g., one or more bioerodable and/or biostable polymers and one or more antimicrobial agents and/or one or more anti-scarring agents, which will exert an antimicrobial action when inserted into a patient, and prevent or reduce or resist protein encapsulation on the surface of the inserted device and associated infections. The materials may also include various polymers which can serve as binders for the agents, and which can mediate the diffusion of such agents from the coating in suitable elution profiles.

In an exemplary embodiment, such polymers may be bioabsorbable. The deciduous nature of bioabsorbable polymeric materials may bias the surface toward protein absorption resistance. The polymer(s) also may contribute to the anti-protein absorbing properties of the surface of the treated device.

In another exemplary embodiment, the invention may be used for preventing microbial infections and protein absorption or encapsulation. Protein absorption or encapsulation is the result of the body's natural process of encapsulating a foreign substance, such as a device as described above, in order to protect the body. The resulting tissue reaction interferes or impedes device function, e.g., insulin absorption or blood sugar monitoring, resulting in the need to replace the device in shorter periods of time. By providing a device with a surface layer having anti-protein absorption and antimicrobial (anti-infectious) characteristics, the incidence of unwanted protein encapsulation and susceptibility to infection is reduced, allowing the device to remain patent and effective for longer periods of time. The advantageous extended patency of the inventive devices means that the devices may remain inserted and effective for their intended purpose (e.g., infusion, draining, sensing or eluting) for substantially longer periods of time than devices without such a surface or coating. Generally, it has been observed and understood by those skilled in the art that needles without such coatings require replacement every two to four days because infections may set in after 2 to 4 days and/or protein absorption/encapsulation may set in after 2 to 5 days. Substantially longer patency may mean an increase of 10% to four fold, or of 1 to 7 days. It can be a period that is longer by at least about 25%, 50%, 75% or double or triple the period for a comparably uncoated device, or at least a day, two days, three days, four days, five days, a week or 10 days longer.

Thus, for example, the inventive devices allow diabetic patients to use only about 52 infusion needles in a year, as opposed to 100-180. This is a significant improvement in comfort, safety, cost and convenience.

As used herein the terms “bioerodable” and “bioabsorbable” materials, e.g., polymer or polymeric surface layer, have similar meaning, namely that they are dissolved or otherwise broken down during insertion or implantation in a patient. In contrast, non-bioabsorbable, insoluble, and biostable materials typically do not dissolve or break down in biological media. The term biocompatible implies that the material does not induce an adverse response when exposed to living tissue other than absorbing proteins and/or other absorbing biological specimens. The term deciduous suggests sloughing off when exposed to body fluids and/or tissue and refers to an appropriate degree of bioerodability and/or bioabsorbability. Bioerodability implies that the material will safely degrade and erode away in living tissue/fluid. The process can be fairly rapid as with water-soluble polymers, or can take place over a more extended time period when the process depends on a hydrolysis reaction(s), e.g., as would be the case with polyglycolic acid esters. Effective sloughing off may occur more with more water-soluble polymers, and less with the polymers that dissolve more slowly, e.g., dispersible polymers. On the other hand, some polymers may have surface characteristics that resist protein absorption by mechanisms other than sloughing off of surface molecules in tissue/fluids, and as such are included in this invention. For example, the anti-protein absorption agent may be a biostable polymer and an anti-scarring, anti-fibrosis or anti-cancer agent.

As used herein, anti-protein absorption agents are those that resist or prevent the absorption or encapsulation of proteins on the device, which may impede device function. For example, those components, e.g., bioerodable polymers, may enable the surface of the device to be deciduous, i.e., to slough off and clear the unwanted absorbed protein from the device surface.

As used herein, the term patency-extending polymeric surface layer refers to a surface layer of a device comprising an anti-protein absorption agent, e.g., polymer or polymer mixture, and anti-infective agent that extend the patency of the devices when inserted into a patient. In an exemplary embodiment, the surface layer may include a polymeric binder of one or more polymers that can serve as binders for the agents, and which can mediate the diffusion of such agents from the coating in suitable elution profiles.

As used herein the term “polymer” may be one or a mixture of two or more polymers. In an exemplary embodiment, the polymer may be bioerodable/bioabsorbable or biostable, for example, the polymer may be a bioerodable polymer. In certain aspects, the polymer may prevent the absorption of proteins onto the device surface, thereby resisting or reducing protein encapsulation of the device. In other aspects, the polymer may slough off from the device, thereby removing absorbed protein from the device surface. The polymer, which may prevent or reduce absorption of proteins onto the surface of the device, may be combined with a therapeutic agent (e.g., an anti-infective agent), such as to provide controlled or sustained release of the agent from the binder. “Release of an agent” can be measured as a statistically significant presence of the agent, or a subcomponent thereof, which has disassociated from the implant/device.

The bioerodable polymers may be water-soluble or dispersible polymers or non-water-soluble polymers that erode via a hydrolytic erosion process. Examples of bioerodable polymers may include polyethylene glycol, polyethylene oxide, acrylic acid or a salt or a copolymer thereof, acrylic emulsion copolymer, a polymer or copolymer of polylactic acid, a polymer or copolymer of polyglycolic acid, polyacrylamide, polyvinylpyrrolidone, polyurethane, water-soluble cellulose polymer, cellulose acetate phthalate, and polyvinylalcohol.

The present invention may comprise a coating composition (e.g., a pre-coating solution or formulation) for coating a device. The coating composition may include a bioerodable polymer at a concentration from about 0.5 to 25%, or from about 5 to 20%, 1 to 10%, 2 to 8%, 3 to 7%, 5 to 6%, 2 to 4%, 4 to 6%,1%, 2%, 3%, 4%, 5%, 6%, 7%, 8%, 9%, 10%, 15%, 20% or 25%. The composition may be applied to the device as one layer or in multiple layers. For example, the composition may be applied as a primer layer, a basecoat layer, and/or a topcoat layer.

In an exemplary embodiment, the present invention may comprise a device with a surface layer comprising bioerodable polymer from about 50% to 99.9%, or from about 70 to 99%, 73 to 97%, 75 to 95%, 80 to 90%, 73%, 80%, 86%, 87%, 89%, 94%, or 97%.

As shown in the Examples, the ratio of anti-infective agent to polymer (in dry weight) in surface layer may vary depending on the strength of the anti-infective and the characteristics of the polymer. Exemplary drug to polymer ratios include 3:97, 5:95, or 6:94, for 5-flurouracil and PEG, and 10:90, 20:80 or 30:70 for 2-bromo-2-nitropropane-1,3-diol (BRONOPOL), Irgasan (TRICLOSAN), and/or polyhexamethylene biguanide (BAQUACIL) and PEG. Thus, the ratio of drug to polymer in the surface layer may be from about 1:99 to 1:2.

In another aspect of the invention the bioerodable polymer comprises polyethylene glycol (PEG) having a high molecular weight of at least about 3500. In another aspect, the molecular weight may be from about 3500 to about 35000. Specific weight ranges may include about 3500, 3500-4500, 4000, 4500, 5000, 5500, 6000, 7000-9000, 7000, 8000, 9000, 10000, 11000, 12000, 13000, 15000, 16000-24000, 20000, 30000, or 35000. Available commercial PEG products may be used with the present invention, for example those marketed by SIGRAMSA-ALDRICH, e.g., product numbers 95904 (MW 3500-4500), 81253 (MW 6000), 81255 (MW 6000), 89510 (MW 7000-9000), 81268 (MW 7000-9000), P2139 (MW 8000), P5413 (MW 8000), P4463 (MW 8000), P5667 (MW 10000), 92897 (MW 8500-11500), 95172 (16000-24000) or 94646 (35000).

In another exemplary embodiment, the bioerodable polymer may comprise a copolymer of methylpolyethylene and poly CD,L-lactic acid (MePEG-PDLLA 60:40). This copolymer is in the class of poly(alkylene oxide)-poly(ester) block copolymers (e.g., X-Y, X-Y-X, Y-X-Y, R—(Y-X)ₙ, or R—(X-Y)ₙ, where X is a polyalkylene oxide (e.g., poly(ethylene glycol, poly(propylene glycol) and block copolymers of poly(ethylene oxide) and polypropylene oxide) (e.g., PLURONIC and PLURONIC R series of polymers from BASF Corporation, Mount Olive, N.J.) and Y is a polyester, where the polyester may comprise the residues of one or more of the monomers selected from lactide, lactic acid, glycolide, glycolic acid, e-caprolactone, gamma-caprolactone, hydroxyvaleric acid, hydroxybutyric acid, beta-butyrolactone, gamma-butyrolactone, gamma-valerolactone, γ-decanolactone, δ-decanolactone, trimethylene carbonate, 1,4-dioxane-2-one or 1,5-dioxepan-2one (e.g., PLGA, PLA, PDLLA, PCL, polydioxanone and copolymers thereof) and R is a multifunctional initiator), and where n can be 2 to 12. Compositions comprising blends of one or more of these polymers may also be used.

In another exemplary embodiment of the invention, the surface layer or composition further comprises a non-bioabsorbable or biostable polymer. Examples of non-bioabsorbable or biostable polymers include acrylates, urethanes, polycarbonates, polyamides, polyesters and polyimides, or a biostable polymer, e.g., cellulose ester polymers and copolymers, insoluble polyurethanes, polyvinyl chloride, polyamides, acrylate polymers and copolymers, ethylenevinylacetate copolymers, vinylpyrrolidoneethylacetate copolymers, acetal polymers and copolymers, silicone polymers and copolymers, polyesters, polyimides and copolymers and polyetherimides. The biostable polymers may harden and help stabilize other components of the surface or coating, without interfering with the character of the outer surface. In another aspect, the non-bioabsorbable or biostable polymer comprises one or more polymers of styrene isobutylene styrene polymers cellulose esters, and/or polystyrene, alkylated polyvinylpyrrolidone.

In one exemplary aspect, the inventive surface layer or coating composition may comprise biostable cellulose esters, e.g., nitrocellulose, insoluble polyurethanes, e.g., those that do not undergo hydrolytic scission in vivo, or acrylic polymers, e.g., ones that are not water soluble or water swellable.

In an exemplary embodiment, an amount of nitrocellulose of up to about 10% of the PEG amount can be used in a coating composition containing the solvent acetonitrile to help enhance the durability of the PEG in the coating.

In another exemplary embodiment, a coating composition or surface layer may comprise a mixture of two or more bioerodable and/or biostable polymers. For example, the surface layer or coating composition may have a polymer mixture of 0.1% nitrocellulose and 99.9% polyethylene glycol (PEG); from about 14 to 18% MePEG-PDLLA 60:40 copolymer and from about 86% to 82% PEG, respectively; 23% epoxy resin, 38% polyurethane resin and 39% polyethylene-co-acrylic acid polymer; or 4.9% melamine-formaldehyde resin, 12.7% polyurethane, 13.3% acrylic polymer and 69% ¼ sec. RS Nitrocellulose (70% nitrocellulose and 30% isopropanol).

In an exemplary embodiment a coating composition or surface layer may further comprise from about 0.02% to 10% nitrocellulose, 0.02% to about 0.1%, or 1% to about 10% of the polymer mixture, of the composition or of the surface layer. A coating composition may comprise nitrocellulose in ethanol, tetrahydrofuran, and benzyl alcohol in a ratio of 2:15:1 by weight.

The surface layer or coating composition may further comprise a polymer, copolymer, polymer or copolymer mixture, resin, epoxy and/or mixtures thereof. For example, the coating composition may comprise one or more of 5% polyethylene-co-acrylic acid polymer, 37% w/w epoxy resin in THF, polyurethane resin 25% w/w in DMA (AR CHLOROFLEX), melamine-formaldehyde resin (CYMEL 248-08 FROM CYTEC), acrylic polymer, polyurethane resin, and/or MePEG/PDLLA 60/40.

Sites within the body that can be accessed by the device include but are not limited to vascular, percutaneous and subcutaneous sites, body cavities, potential spaces, pathologic cavities, and other sites accessible through the dermis layer of the skin. Depending on the purpose of the device and/or the environment and point of insertion or place of implantation, the extent of protein absorption and/or susceptibility of microbial infection may differ. Based on the level of protein absorption and/or susceptibility of microbial infection and the type of tissue environment, the amounts and types of components of the anti-protein absorption and anti-microbial/infectious surface layer may be adjusted to either reduce or increase the amount and rate at which the coat can slough off. For example, for catheters placed into blood vessels, where fluid flow increases the erosion of the coating, a more durable surface may be required.

In one aspect, the device may be a needle that is inserted intradermally or a catheter that is implanted vascularly. In one embodiment, the device may be a 26 gauge insulin pump needle that is inserted intradermally (e.g., Bent Needles from Medtronic MiniMed) and a portion of the needle may contain the anti-protein absorption and anti-microbial/infectious surface layer, e.g., 1.5 cm, where 1.0 to 1.5 cm of the device is inserted. The exterior portion of the needle may be taped down using the disc described below. The needle may be connected to a delivery tube that is connected to an insulin pump, e.g., a 3 ml syringe reservoir that may be filled with insulin.

The inventive surface layer may comprise an agent which inhibits infection. “Inhibit infection” refers to the ability of an agent or composition to prevent microorganisms from accumulating and/or proliferating near or at the site of the agent. An agent which inhibits infection is referred to herein as an “anti-infective agent” or “anti-microbial agent.” Anti-infective agents include those compounds capable of combating infections resulting from a variety of sources (e.g., bacterial, viral, fungal, and the like). These processes would be expected to occur at a statistically significant level at or near the site of the agent or composition relative to the effect in the absence of the agent or composition.

Representative examples of antimicrobial (anti-infective) agents include a quaternary compound, a phenolic compound, an iodinated compound, a silver compound or an acidic-anionic compound. Examples of anti-infective agents include one or more of 2-bromo-2-nitropropane-1,3-diol (e.g., BRONOPOL), Irgasan (TRICLOSAN), polyhexanide (also known as polyhexamethylene biguanide) (e.g., VANTOCIL IB, COSMOCIL CQ, or BAQUACIL), benzalkonium chloride, benzethonium chloride, cetylpyradinium chloride, stearalkonium chloride, phenol, cresol, aminophenol, iodine, iodide, 8-hydroxyquinolone, and chlorhexidine.

Other examples of bioactive agents which have been shown to have anti-microbial (anti-infective) characteristics, in addition to other therapeutic uses, may be used in the present compositions. For example, the anti-infective agent may be a chemotherapeutic agent. Numerous chemotherapeutic agents have been identified, which have potent antimicrobial activity at extremely low doses. Examples of these agents are described in U.S. Published Patent Application No. 20040043052, which is incorporated herein in its entirety, and include anthracyclines (e.g., doxorubicin and mitoxantrone), fluoropyrimidines (e.g., 5-fluorouracil (5-FU)), folic acid antagonists (e.g., methotrexate), podophylotoxins (e.g., etoposide), camptothecins, hydroxyureas, and platinum complexes (e.g., cisplatin), and/or analogs or derivatives thereof.

Exemplary anthracyclines include doxorubicin, daunorubicin, idarubicin, epirubicin, pirarubicin, zorubicin, carubicin, anthramycin, mitoxantrone, menogaril, nogalamycin, aclacinomycin A, olivomycin A, chromomycin A₃, plicamycin, FCE 23762, a doxorubicin derivative, annamycin, ruboxyl, anthracycline disaccharide doxorubicin analog, 2-pyrrolinodoxorubicin, disaccharide doxorubicin analogs, 4-demethoxy-7-O-[2,6-dideoxy-4-O-(2,3,6-trideoxy-3-amino-α-L-lyxo-hexopyranosyl)- α-L-lyxo-hexopyranosyl]adriamicinone doxorubicin disaccharide analog, 2-pyrrolinodoxorubicin, morpholinyl doxorubicin analogs, enaminomalonyl-β-alanine doxorubicin derivatives, cephalosporin doxorubicin derivatives, hydroxyrubicin, methoxymorpholino doxorubicin derivative, (6-maleimidocaproyl)hydrazone doxorubicin derivative, N-(5,5-diacetoxypent-1-yl) doxorubicin, FCE 23762 methoxymorpholinyl doxorubicin derivative, N-hydroxysuccinimide ester doxorubicin derivatives, polydeoxynucleotide doxorubicin derivatives, morpholinyl doxorubicin derivatives, mitoxantrone doxorubicin analog, AD198 doxorubicin analog, 4-demethoxy-3′-N-trifluoroacetyldoxorubicin, 4′-epidoxorubicin, alkylating cyanomorpholino doxorubicin derivative, deoxydihydroiodooxorubicin, adriblastin, 4′-deoxydoxorubicin, 4-demethyoxy-4′-o-methyldoxorubicin, 3′-deamino-3′-hydroxydoxorubicin, 4-demethyoxy doxorubicin analogs, N-L-leucyl doxorubicin derivatives, 3′-deamino-3′-(4-methoxy-1-piperidinyl) doxorubicin derivatives, 3′-deamino-3′-(4-mortholinyl) doxorubicin derivatives, 4′-deoxydoxorubicin and 4′-o-methyldoxorubicin, aglycone doxorubicin derivatives, SM 5887, MX-2, 4′-deoxy-13(S)-dihydro-4′-iododoxorubicin, morpholinyl doxorubicin derivatives, 3′-deamino-3′-(4-methoxy-1-piperidinyl) doxorubicin derivatives, doxorubicin-14-valerate, morpholinodoxorubicin, 3′-deamino-3′-(3″-cyano-4″-morpholinyl doxorubicin, 3′-deamino-3′-(3″-cyano-4″-morpholinyl)-13-dihydoxorubicin, (3′-deamino-3′-(3″-cyano-4″-morpholinyl) daunorubicin, 3′-deamino-3′-(3″-cyano-4″-morpholinyl)-3-dihydrodaunorubicin, 3′-deamino-3′-(4″-morpholinyl-5-iminodoxorubicin, 3′-deamino-3′-(4-methoxy-1-piperidinyl) doxorubicin derivatives, and 3-deamino-3-(4-morpholinyl) doxorubicin derivatives.

Exemplary fluoropyrimidine analogs include 5-fluorouracil, or an analog or derivative thereof, including carmofur, doxifluridine, emitefur, tegafur, and floxuridine. Other exemplary fluoropyrimidine analogs include 5-FudR (5-fluoro-deoxyuridine), or an analog or derivative thereof, including 5-iododeoxyuridine (5-IudR), 5-bromodeoxyuridine (5-BudR), fluorouridine triphosphate (5-FUTP), and fluorodeoxyuridine monophosphate (5-dFUMP). Other representative examples of fluoropyrimidine analogs include N3-alkylated analogs of 5-fluorouracil, 5-fluorouracil derivatives with 1,4-oxaheteroepane moieties, 5-fluorouracil and nucleoside analogs, cis- and trans-5-fluoro-5,6-dihydro-6-alkoxyuracil, cyclopentane 5-fluorouracil analogs, A-OT-fluorouracil, N4-trimethoxybenzoyl-5′-deoxy-5-fluorocytidine and 5′-deoxy-5-fluorouridine, 1-hexylcarbamoyl-5-fluorouracil, B-3839, uracil-1-(2-tetrahydrofuryl)-5-fluorouracil, 1-(2′-deoxy-2′-fluoro-β-D-arabinofuranosyl)-5-fluorouracil, doxifluridine, 5′-deoxy-5-fluorouridine, 1-acetyl-3-O-toluyl-5-fluorouracil, 5-fluorouracil-m-formylbenzene-sulfonate, N′-(2-furanidyl)-5-fluorouracil and 1-(2-tetrahydrofuryl)-5-fluorouracil.

Exemplary folic acid antagonists include methotrexate or derivatives or analogs thereof, including edatrexate, trimetrexate, raltitrexed, piritrexim, denopterin, yomudex, pteropterin. Other representative examples include 6-S-aminoacyloxymethyl mercaptopurine derivatives, 6-mercaptopurine (6-MP), 7,8-polymethyleneimidazo-1,3,2-diazaphosphorines, azathioprine, methyl-D-glucopyranoside mercaptopurine derivatives and s-alkynyl mercaptopurine derivatives, indoline ring and a modified ornithine or glutamic acid-bearing methotrexate derivatives, alkyl-substituted benzene ring C bearing methotrexate derivatives, benzoxazine or benzothiazine moiety-bearing methotrexate derivatives, 10-deazaanminopterin analogs, 5-deazaaminopterin and 5,10-dideazaaminopterin methotrexate analogs, indoline moiety-bearing methotrexate derivatives, lipophilic amide methotrexate derivatives, L-threo-(2S,4S)-4-fluoroglutamic acid and DL-3,3-difluoroglutamic acid-containing methotrexate analogs, methotrexate tetrahydroquinazoline analog, N-(α-aminoacyl) methotrexate derivatives, biotin methotrexate derivatives, D-glutamic acid or D-erythrou, threo-4-fluoroglutamic acid methotrexate analogs, β,γ-methano methotrexate analogs, 10-deazaaminopterin (10-EDAM) analog, γ-tetrazole methotrexate analog, N-(L-α-aminoacyl) methotrexate derivatives, meta and ortho isomers of aminopterin, hydroxymethylmethotrexate, γ-fluoromethotrexate, polyglutamyl methotrexate derivatives, gem-diphosphonate methotrexate analogs, α- and γ-substituted methotrexate analogs, 5-methyl-5-deaza methotrexate analogs, Nδ-acyl-Nα-(4-amino-4-deoxypteroyl)-L-ornithine derivatives, 8-deaza methotrexate analogs, acivicin methotrexate analog, polymeric platinol methotrexate derivative, methotrexate-γ-dimyristoylphophatidylethanolamine, methotrexate polyglutamate analogs, poly-γ-glutamyl methotrexate derivatives, deoxyuridylate methotrexate derivatives, iodoacetyl lysine methotrexate analog, 2-omega-diaminoalkanoid acid-containing methotrexate analogs, polyglutamate methotrexate derivatives, 5-methyl-5-deaza analogs, quinazoline methotrexate analog, pyrazine methotrexate analog, cysteic acid and homocysteic acid methotrexate analogs, γ-tert-butyl methotrexate esters, fluorinated methotrexate analogs, folate methotrexate analog, phosphonoglutamic acid analogs, poly (L-lysine) methotrexate conjugates, dilysine and trilysine methotrexate derivates, 7-hydroxymethotrexate, poly-γ-glutamyl methotrexate analogs, 3′,5′-dichloromethotrexate, diazoketone and chloromethylketone methotrexate analogs, 10-propargylaminopterin and alkyl methotrexate homologs, lectin derivatives of methotrexate, polyglutamate methotrexate derivatives, halogentated methotrexate derivatives, 8-alkyl-7,8-dihydro analogs, 7-methyl methotrexate derivatives and dichloromethotrexate, lipophilic methotrexate derivatives and 3′,5′-dichloromethotrexate, deaza amethopterin analogs, MX068 and cysteic acid and homocysteic acid methotrexate analogs.

Exemplary podophyllotoxins include etoposide, teniposide, Cu(II)-VP-16 (etoposide) complex, pyrrolecarboxamidino-bearing etoposide analogs, 4β-amino etoposide analogs, γ-lactone ring-modified arylamino etoposide analogs, N-glucosyl etoposide analog, etoposide A-ring analogs, 4′-deshydroxy-4′-methyl etoposide, pendulum ring etoposide analogs and E-ring desoxy etoposie analogs.

Exemplary camptothecins include topotecan, irinotecan (CPT-11), 9-aminocamptothecin, 21-lactam-20(S)-camptothecin, 10,11-methylenedioxycamptothecin, SN-38, 9-nitrocamptothecin, and 10-hydroxycamptothecin.

Exemplary platinum complexes include complexes of Pt(II) or Pt(IV), cisplatin, carboplatin, oxaliplatin, and miboplatin. Other representative examples of platinum compounds include (CPA)₂Pt[DOLYM] and (DACH)Pt[DOLYM] cisplatin, Cis-[PtCl₂(4,7-H-5-methyl-7-oxo] 1,2,4[triazolo[1,5-a]pyrimidine)₂], [Pt(cis-1,4-DACH)(trans-Cl₂)(CBDCA)] ●½MeOH cisplatin, 4-pyridoxate diammine hydroxy platinum, Pt(II) ●●●Pt(II) (Pt₂[NHCHN(C(CH₂)(CH₃))]₄), 254-S cisplatin analog, o-phenylenediamine ligand bearing cisplatin analogs, trans, cis-[Pt(OAc)₂I₂(en)], estrogenic 1,2-diarylethylenediamine ligand (with sulfur-containing amino acids and glutathione) bearing cisplatin analogs, cis-1,4-diaminocyclohexane cisplatin analogs, 5′ orientational isomer of cis-[Pt(NH₃)(4-aminoTEMP-O){d(GpG)}], chelating diamine-bearing cisplatin analogs, 1,2-diarylethyleneamine ligand-bearing cisplatin analogs, (ethylenediamine)platinum(II) complexes, CI-973 cisplatin analog, cis-diaminedichloroplatinum(II) and its analogs cis-1,1-cyclobutanedicarbosylato(2R)-2-methyl-1,4-butanediamineplatinum(II) and cis-diammine(glycolato)platinum, cis-amine-cyclohexylamine-dichloroplatinum(II), gem-diphosphonate cisplatin analogs, (meso-1,2-bis(2,6-dichloro-4-hydroxyplenyl)ethylenediamine) dichloroplatinum(II), cisplatin analogs containing a tethered dansyl group, platinum(II) polyamines, cis-(3H)dichloro(ethylenediamine)platinum(II), trans-diamminedichloroplatinum(II) and cis-(Pt(NH₃)₂(N₃-cytosine)Cl), 3H-cis-1,2-diaminocyclohexanedichloroplatinum(II) and 3H-cis-1,2-diaminocyclohexanemalonatoplatinum (II), diaminocarboxylatoplatinum, trans-(D,1)-1,2-diaminocyclohexane carrier ligand-bearing platinum analogs, aminoalkylaminoanthraquinone-derived cisplatin analogs, spiroplatin, carboplatin, iproplatin and JM40 platinum analogs, bidentate tertiary diamine-containing cisplatinum derivatives, platinum(II), platinum(IV), cis-diammine(1,1-cyclobutanedicarboxylato-)platinum(II) (carboplatin, JM8) and ethylenediammine-malonatoplatinum(II) (JM40), JM8 and JM9 cisplatin analogs, (NPr4)2((PtCL4).cis-(PtCl12-(NH2Me)2)), aliphatic tricarboxylic acid platinum complexes, and cis-dichloro(amino acid)(tert-butylamine)platinum(II) complexes.

In one embodiment, the anti-infective agent may be benzalkonium heparinate or sodium heparin. In another aspect of the invention, the surface layer does not contain any ethylenediamine tetraacetic acid (EDTA).

The present invention may comprise a surface layer comprising antimicrobial (anti-infective) agents from about 0.1% to 50%, or from about 0.5% to 30%, 3% to 27%, 3%, 6%, 11%, 13%, 17%, 20%, 25% or 27% by weight.

In an exemplary embodiment, the device may be a coated infusion needle (e.g., 27 gauge needle about 1.5 cm long) and may include antimicrobial (anti-infective) agents in an amount of about 0.5 to about 5 micrograms; or about 5 to about 10 micrograms; or about 10 to about 20 micrograms. In one aspect, the device may be a hand-coated needle comprising about 0.65, 1.20 or 4.34 micrograms of anti-infective agent. In other examples, the amounts or concentrations of anti-infective agent may be substantially lower or higher.

The present invention may comprise a composition, formulation or solution (pre-coating) for coating a device that includes antimicrobial (anti-infective) agents at a concentration from about 0.01 to 8.0%, 0.5 to 5.5%, 0.01 to 1.4%, 0.1-2%, 0.2-1.0%, 0.2%, 0.3%, 0.4%, 0.5%, 0.6%, 0.7%, 0.8%, 0.9%, 1.0%, 1.5%, 2.0%, 2.5%, 3.0%, 3.5%, 4.0%, 4.5%, 5.0%, or 5.5% by weight. The composition may be applied to the device in multiple layers, e.g., primer, basecoat or topcoat.

In an exemplary embodiment, the surface layer may comprise chemotherapeutic, antimicrobial (anti-infective) agents including but not limited to: anthracyclines (e.g., doxorubicin and mitoxantrone), fluoropyrimidines (e.g., 5-FU), folic acid antagonists (e.g., methotrexate), podophylotoxins (e.g., etoposide), camptothecins, hydroxyureas, and platinum complexes (e.g., cisplatin), and/or analogs or derivatives thereof. For example, such agents may be used in amounts that range from about 50% to 30%, 20%, 10%, 5%, or even less than 1% of the amount typically used in a single chemotherapeutic systemic dose application.

In certain aspects, the anti-infective compound may be released from the device. In one embodiment, the drug can be released in effective concentrations for a period ranging from 1 to 30 days. In another exemplary aspect, the agents may be included as follows: total dose not to exceed 10 mg (range of 0.1 μg to 10 mg), e.g., 1 μg to 3 mg; dose per unit area of the device of 0.1 μg-30 μg per mm², e.g., dose of 0.25 μg/mm²-20 μg/mm²; and/or minimum concentration of 10⁻⁸-10⁻³ M of drug is to be maintained on the device surface for a period from one to thirty days.

The inventive solution, formulation or composition (pre-coating) for coating the surface layer may further comprise a solvent. Suitable solvents include those that are compatible with the anti-infective and/or the anti-protein absorption agent, and are appropriate for human use as residues in the coating. In an exemplary embodiment, the solvent may be selected from solvents that are able to dissolve or disperse the components homogeneously. Examples of solvents include one or more of the following: water, acetonitrile, methylethyl ketone (MEK), denatured ethanol, ethyl alcohol (ethanol), saline solution, normal saline solution, tetrahydrofuran (THF), isopropyl alcohol (isopropanol), other alcohols, amines, amides, 1,3-dioxalane, ketones, esters, cyclic compounds, glycols, carboxylic acids or aromatic solvents. In another exemplary embodiment, the solvent may be cyclohexanone, toluene, benzyl alcohol, dibutylphthalate, butanol, xylene and/or ethyl benzene.

The solvent may be an aqueous or an organic solvent. The composition may comprise from about 50% to about 99% or from about 70% to 99%, 70% to 80%, 80% to 90%, or 90% to about 98.8% solvent. In one aspect of the invention, the composition comprises one or more solvents, e.g., water, methylethyl ketone, tetrahydrofuran, 1,3-dioxalane isopropyl alcohol, acetonitrile or denatured ethanol.

In another exemplary embodiment, the inventive surface layer, composition or solution may further include buffers, colorants, surfactants and other components that are biocompatible and do not interfere with the other components in the composition. An example of a surfactant is Tween 80, e.g., 1.00% w/w Tween 80 aq. Examples of colorants may include Gentian Violet (Hucker Formula) and/or dimethylmethylene blue. In another exemplary embodiment, Gentian Violet (Hucker Formula) may be used as an anti-infective agent.

The inventive surface layer, composition or solution may further comprise a therapeutic agent (referred to synonymously herein as a drug or bioactive agent). These agents may be incorporated into the coating composition. In one exemplary embodiment, the surface layer may comprise one or more of bactericides, antibiotics, antiviral, antiseptics, antineoplastics, anticancer compounds, antifungal, and anti-yeast and/or anti-fibrosis or anti-scarring agents (e.g., mycophenoloic acid), or other bioactive or therapeutic agents that are suitable for human use. The surface layer or composition may comprise from about 0.01 to 8.0% or 0.5 to 5.5% for each of the above agents.

In one aspect, the surface layer may comprise a therapeutic agent that inhibits fibrosis or scarring. “Fibrosis,” or “scarring,” or “fibrotic response” refers to the formation of fibrous (scar) tissue in response to injury or medical intervention. Therapeutic agents which inhibit fibrosis or scarring are referred to herein as “fibrosis-inhibiting agents”, “anti-fibrosis agents”, “fibrosis-inhibitors”, “anti-scarring agents”, and the like, where these agents inhibit fibrosis through one or more mechanisms including: inhibiting inflammation or the acute inflammatory response, inhibiting migration or proliferation of connective tissue cells (such as fibroblasts, smooth muscle cells, vascular smooth muscle cells), inhibiting angiogenesis, reducing extracellular matrix (ECM) production or promoting ECM breakdown, and/or inhibiting tissue remodeling.

For example, anti-scarring or fibrosis inhibiting agents may be incorporated to improve the function of the device e.g. enhancing resistance to protein absorption. Representative examples of fibrosis inhibiting agents which can inhibit pathological processes in the treatment site include, but not limited to, the following classes of compounds: anti-inflammatory agents e.g., dexamethasone, cortisone, fludrocortisone, prednisone, prednisolone, 6α-methylprednisolone, triamcinolone, and betamethasone), MMP inhibitors (e.g., batimistat, marimistat, and TIMP's); cytokine inhibitors (e.g., chlorpromazine, mycophenolic acid, rapamycin, 1α-hydroxy vitamin D₃), IMPDH (e.g., inosine monophosplate dehydrogenase) inhibitors (e.g., mycophenolic acid, ribaviran, aminothiadiazole, thiophenfurin, tiazofurin, viramidine), p38 MAP kinase inhibitors (MAPK) (e.g., GW-2286, CGP-52411, BIRB-798, SB220025, RO-320-1195, RWJ-67657, RWJ-68354, SCIO-469), and immunomodulatory agents (rapamycin, everolimus, ABT-578, azathioprine azithromycin, analogs of rapamycin, including tacrolimus and derivatives thereof and everolimus and derivatives thereof, and sirolimus and analogs and derivatives thereof (e.g., ABT-578).

In one aspect, agents that inhibit fibrosis include paclitaxel, sirolimus, everolimus, vincristine, biolimus, ABT-578, cervistatin, simvastatin, methylprednisolone, dexamethasone, actinomycin-D, angiopeptin, L-arginine, estradiol, 17-β-estradiol, tranilast, methotrexate, batimistat, halofuginone, BCP-671, QP-2, lantrunculin D, cytochalasin A, nitric oxide, and analogs and derivatives thereof.

Other exemplary drugs that may be included in the surface layer, compositions and devices of the invention include tyrosine kinase inhibitors, such as imantinib, ZK-222584, CGP-52411, CGP-53716, NVP-AAK980-NX, CP-127374, CP-564959, PD-171026, PD-173956, PD-180970, SU-0879, and SKI-606. Other examples of MMP inhibitors include nimesulide, PKF-241-466, PKF-242-484, CGS-27023A, SAR-943, primomastat, SC-77964, PNU-171829, AG-3433, PNU-142769, SU-5402, and dexlipotam; p38 MAP kinase inhibitors such as CGH-2466 and PD-98-59; immunosuppressants such as argyrin B, macrocyclic lactone, ADZ-62-826, CCI-779, tilomisole, amcinonide, FK-778, AVE-1726, and MDL-28842; and cytokine inhibitors such as TNF-484A, PD-172084, CP-293121, CP-353164, and PD-168787. Other examples include NFKB inhibitors, such as, AVE-0547, AVE-0545, and IPL-576092 and HMGCoA reductase inhibitors, such as, pravestatin, atorvastatin, fluvastatin, dalvastatin, glenvastatin, pitavastatin, CP-83101, U-20685, apoptosis antagonists (e.g., troloxamine, TCH-346 (N-methyl-N-propargyl-10-aminomethyl-dibenzo(b,f)oxepin), caspase inhibitors (e.g., PF-5901 (benzenemethanol, alpha-pentyl-3-(2-quinolinylmethoxy)-), and JNK inhibitor (e.g., AS-602801).

In another embodiment, the surface layer, composition or solution may further comprise a corticosteroid, such as synthetic or natural corticosteroids, e.g., dexamethasone, alclometasone dipropionate, amcinonide, betamethasone, clobetasol proprionate, clocortolone pivalate, cortisone, hydrocortisone, desonide, desoximetasone, diflorasone diacetate, fluocinolone acetonide, fluocinonide, fluandrenolide, halcinonide, methylprednisolone, mometasone furoate, and triamcinolone.

In another embodiment, the surface layer, composition or solution may comprise a non-steroidal anti-inflammatory drug (NSAID), such as aspirin, phenylbutazone, indomethacin, sulindac, tolmetin, ibuprofen, piroxicam, fenamates, acetaminophen and phenacetin.

In another embodiment of the invention, the composition or solution may be applied onto the surface in the form of a coating, or the surface layer may comprise two or more coating layers, e.g., a primer, basecoat or topcoat. For example, the primer may be the layer that binds to the substrate (e.g., stainless steel) of the device, the basecoat may be a layer whose presence stabilizes the outermost layer to the primer layer or device surface, and the topcoat, e.g., polymer/drug-containing or releasing layer, may be the outermost layer.

In one aspect of the invention, the primer composition comprises at least one or more solvents and at least one biostable polymer or resin, e.g., 5% polyethylene-co-acrylic acid polymer, 37.5% w/w Epoxy resin in THF and polyurethane resin 25% in DMA.

In another aspect the basecoat composition comprises at least one or more solvents and at least one bioerodable and/or a biostable polymer or resin. The basecoat composition may comprise about 70% to 90% solvent and about 10% to 20% polymer or resin. The basecoat composition may comprise solvents such as acetonitrile, denatured ethanol and methylethyl ketone, and polymers such as nitrocellulose and polyethylene glycol 8000. In another exemplary embodiment, the basecoat composition may comprise solvents such as toluene, benzyl alcohol, tetrahydrofuran (THF), cyclohexanone, dibutylphthalate, butanol, xylene and ethylbenzene and polymers or resins such as melamine-formaldehyde resin, acrylic polymer, nitrocellulose and polyurethane resin.

In an exemplary aspect, the topcoat comprises at least a solvent, an anti-infective agent and at least one polymer, which can be bioerodable. In another exemplary aspect, the topcoat composition comprises about 70 to 90% solvent and about 10 to 30% bioerodable polymer. The topcoat composition may comprise solvents such as water, isopropyl alcohol, ethanol and acetonitrile and bioerodable polymers such as MePEG/PDLLA 60/40 and polyethylene glycol 20000.

In another exemplary embodiment, the primer, basecoat and/or topcoat composition may contain at least one polymer and at least one anti-infective agent.

The present invention also provides a kit useful for preventing or inhibiting protein absorption and development of infections arising from insertion or implantation of a medical device through a bodily surface. The kit may comprise an insertable medical device and a disc (cuff). The device has a portion that can be inserted or implanted into the body. A portion of, or the entire surface of the insertable device may comprise an inventive surface layer or a coating that resists protein absorption and formation of infections on the surface of the device. The disc is capable of being penetrated by the device. Alternatively, the disc may be provided with an aperture of suitable size and shape to accommodate passage of the anti-infective, and anti-protein absorbing medical device. Moreover, the disc can be placed around the device post insertion. In use, the disc should be in contact with the body surface and surrounds and abuts the portion of the insertable portion of the device at the point where it projects from the surface of the body.

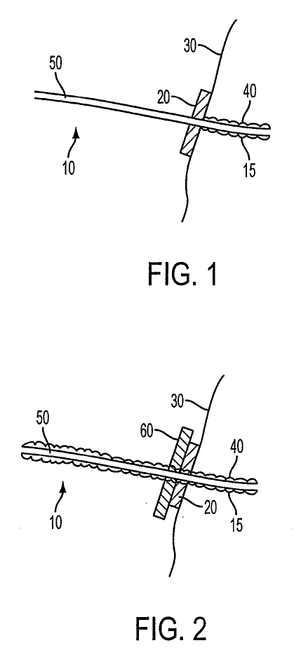

An example of the invention is set forth in FIG. 1, wherein the kit comprises an insertable medical device 10 and a disc 20. The insertable medical device 10 is capable of penetrating or passing through a body surface 30. The device comprises a distal portion 40 that is capable of being inserted or implanted into the body and a proximal portion 50 that remains outside the body.

The disc can be used with any insertable or implantable medical device. The disc can be provided with anti-microbial properties by being coated or saturated with an antimicrobial composition. An exemplary composition may comprise at least one antimicrobial agent capable of exhibiting antimicrobial activity when essentially dry or when solvated after being essentially dry.

Another aspect of this invention provides a kit comprising an insertable medical device and disc as well as a swab, wetted with a coating solution that contains agents intended to resist protein absorption and infectious formations. The swab preferably is used to coat the insertable portion of the device, before the device is placed into the body. The kit of the invention can also include an absorbent pad wetted with a composition containing agents intended to resist protein absorption and infectious formations. The insertable medical device can be placed into subcutaneous tissue, a peripheral vein, a central vein, an artery, a physiologic body cavity or a pathologic cavity.

The disc can have a sufficient amount of adhesive on one surface to adhere the disc to the body surface and can be flexible, porous and/or absorbent. Examples of materials that the disc can be composed of are polypropylene, polyethylene, and woven materials composed of polyester, rayon or cotton.

In one embodiment of the invention, the disc comprises at least two layers. A first layer can be placed against the body surface, and preferably is permeable to the antimicrobial agent(s). A second layer preferably contains an antimicrobial agent in a solvated or dry form, such that the antimicrobial agent can permeate through the first layer.

The invention includes a method of inhibiting or reducing the incidence of protein absorption and infection associated with inserting a medical device in a patient, wherein an insertable surface of said device is coated, at least in part, with a coating that renders said coated surface resistant to protein absorption and infectious formation, which comprises inserting the device in a patient such that a portion of an inserted surface of the device projects from a bodily surface. A disc may be contacted with the bodily surface where the device projects from the bodily surface such that said disc surrounds and abuts the inserted device projecting from the bodily surface, wherein the disc is coated or saturated with an antimicrobial composition. The composition comprises at least one antimicrobial agent capable of exhibiting antimicrobial activity when in a substantially dry state or when solvated after being in a substantially dry state.

In an exemplary embodiment, the outer surface of the distal portion 40 of the insertable medical device (the inserted portion) may be coated with a coating 15 that resists protein absorption and infectious formation. The coating may cover part of the device, as shown in FIG. 1, or its entire surface as shown in FIG. 2. Optionally, the proximal portion 50 of the device is coated with an anti-protein absorption, anti-infective coating. In another aspect, the device lumen may also be coated over part or all of its length.

The anti-protein absorption, anti-infective coating is capable of reducing or eliminating infectious contamination that occurs during the introduction of the device into the body and has anti-protein absorption, antiseptic, antibiotic, disinfectant, antiviral, and/or antifungal properties. In one embodiment of the invention, a swab wetted with the anti-protein absorption, anti-infective composition optionally is provided so that wiping the device with the swab and allowing it to dry before insertion can coat the device, and thereby producing an embodiment of the inventive surface.

An aspect of this invention provides a kit comprising an insertable medical device and disc, wherein said medical device is provided with a treatment that produces a device that exhibits resistance to protein absorption and formation of infections on the surface of the inserted medical device. The insertable medical device has the treatment that resists protein absorption and formation of infections deposited on at least a portion of the device surface, preferably on some of the portion that is inserted into a patient, and more preferably on at least the entire inserted surface of the device, or on the entire surface of the device. Such treatment could consist of a coating that contains agents and or materials that provide the device with both anti-infective and anti-protein absorbing properties. Materials include but are not limited to compounds that exert specific actions such as disinfecting materials, antibiotics, antineoplastics, and other compounds that are known to exert one or more specific physiological actions.

Referring again to the figures, the disc 20 is substantially planar and is composed of an absorbent or non-absorbent material, preferably, an absorbent material. Examples of appropriate materials include, but are not limited to, plastic foams, cotton gauzes, or porous filter material, polypropylene film, polyethylene film, and woven materials composed of polyester, rayon or cotton. As used herein, the term disc includes an object having a surface capable of contacting a bodily surface, regardless of the actual shape. In practice the disc 20 can be circular, rectangular, or any other suitable shape. Hence, the disc 20 is of a shape and size appropriate to the type of medical device and the location where the device 10 is placed. For example, a larger bore access device may require a larger disc 20 than a smaller bore device. A circular disc 20 with a diameter of approximately 2.5 cm can be used for a small needle device. A peritoneal dialysis catheter may require a substantially larger disc 20 measuring up to 15 cm in size and preferably rectangular in shape.

As shown in FIG. 2, another embodiment of the present invention comprises an absorbent pad 60 used in combination with a coated disc 20 to form the disc. The coated disc 20 preferably is composed of a flexible inert material. Suitable materials include but are not limited to polypropylene film and polyethylene film, woven materials composed of polyester, rayon and cotton. The coated disc can be rendered permeable by the presence of a multitude of fine perforations. The fine holes permit easier penetration of the disc 20 by the insertable portion of the device 10. The holes allow access of the solution contained within the disc 20 to the body surface 30, and also allow drainage of any exudates or transudate from the body surface entry site, which can solvate the dried anti-infective composition permitting it to exert its anti-infective properties at the site where the insertable medical device 10 enters the body. The absorbent pad 60 is composed of a material capable of absorbing or being soaked or wetted by the antimicrobial composition. Examples of appropriate materials include, but are not limited to, plastic foams, cotton gauzes, or porous filter material.

The disc 20 may have an anti-infective coating applied to one or both sides of the disc 20 and allowed to dry, so that the disc 20 preferably is dry when applied to the skin. Disc 20 may be of approximately the same size and shape as the absorbent pad 60. However, the absorbent pad 60 and coated disc 20 also can have different sizes and shapes. Optionally, the disc and pad may be adhered to one another. In addition, the disc 20 may be provided with an adhesive material at one surface that permits the disc to adhere to the body surface 30. In use, the disc 20 preferably contacts the body surface 30. The absorbent pad 60 preferably contacts the disc 20, separated from the body surface 30 by the coated disc 20. If only one side of the disc is coated with antimicrobial composition, the coated side preferably is placed against the body surface 30, although it has been found that the perforations in the disk enable the antimicrobial agent(s) in the coating to reach the skin surface even if the disc is placed such that the coating is on the side away from the skin. The system may be secured to the skin with an adhesive material such as adhesive tape.

The disc 20 can be coated, impregnated or saturated or otherwise provided with an antimicrobial composition with antiseptic, antibiotic, disinfectant, antiviral, and/or antifungal properties. An amount of antimicrobial coating is provided to the disc, which is sufficient to provide an effective amount of the antimicrobial agent, when the disc is exposed to moist skin flora or exudate from the puncture site.

The disc 20 preferably surrounds and abuts the insertable portion of the device 10 at a position on device 10 where a portion of the device 10 projects from the body surface 30. In one embodiment of the invention, the disc 20 is placed onto the body surface 30 and the insertable portion of the device is then passed through the disc 20 into the body. In another embodiment, the insertable portion of the device is passed through the center of the disc 20, and is then inserted into the body. In a third embodiment, the kit is packaged with the disc 20 already in place on the device 10. In another embodiment of the present invention the disc 20 has an opening or slit extending from a radially interior portion to its edge. In this embodiment, the disc 20 is placed on the body surface 30, around the device 10 after the device 10 has been inserted into the body.

The disc 20 preferably is dry when applied to body surface 30 and when the device 10 is inserted into the body. If an exudate develops at the access site, it can be absorbed by the disc 20. The exudate can solubilize or solvate the anti-infective material, which can exert an anti-infective effect at the site, limiting or preventing infection. In one embodiment of the invention, only the disc 20 is supplied for use with the medical access device 10 of the user's choice. Optionally, a swab wetted with the anti-infective coating can be supplied for coating the selected medical access device.

In another exemplary embodiment, the invention provides a kit for reducing protein absorption and development of infections arising from insertion of a medical device through a body surface comprising: a) an insertable medical device having a percutaneously insertable surface, b) means for providing the insertable surface with an anti-infective, anti-protein absorption coating, wherein the coating comprises at least one anti-infective agent and at least one polymer; and c) a disc comprising at least one anti-infective agent, said disc being adapted to surround and abut said percutaneously insertable surface when the device is inserted in a subject and a portion of said percutaneously insertable surface projects from an external bodily surface of the subject, and said disc is in contact with said external bodily surface of the subject. The means for providing the coating may be a coating formed on the needle or a swab or an absorbent pad having a composition comprising at least one anti-infective agent at least one polymer. The device, the disc, and/or the swab or the absorbent pad may be packaged together or packaged separately. The disc, the swab, and/or the absorbent pad may be saturated with a composition comprising at least one anti-infective agent and at least one polymer.

The subject may be a human or a non-human animal. In another aspect, the device may be uncoated and the swab may be wetted with a composition comprising at least one anti-infective, anti-protein absorption agent for coating the surface of the device.

In yet another exemplary embodiment, the invention provides a method of coating an insertable medical device, comprising applying a coating comprising a composition comprising at least one anti-infective agent and at least one polymer, either by (a) applying the coating prior to packaging the device or (b) coating the device with a moistened swab or pad after removing the device from its package prior to insertion. The coating may be applied by spraying, dipping or wiping or may be manufactured using an extrusion process. The coating may be applied and then dried at an elevated temperature. For example, the device may be coated with the composition and then dried by heating, e.g., an oven or a blow dryer, at a temperature of at least about 40 degrees Celsius, 40 to 100 degrees Celsius, 40 to 90 degrees Celsius, 40 to 60 degrees Celsius, or about 40, 50, 60, 70, 80 or 90 degrees Celsius.

The invention also provides a method of extending the patency (average insertion time without obstruction) of an insertable medical device comprising providing a coating comprising at least one anti-infective agent and at least one polymer, which may be bioerodable. The coating may reduce the incidence and/or severity of protein absorption and build up and/or the incidence and/or severity of infections occurring at or associated with the site of insertion of the device. In an exemplary aspect, the device is inserted and remains patent for at least about 5 days or longer, e.g. 5 to 10 days, 6 to 9 days, 7 to 8 days, 6 days, 7 days, 8 days, 9 days or 10 days.

The invention provides a method of using an insertable medical device coated with a composition comprising at least one anti-infective agent and at least one polymer, comprising inserting the device into a subject. In one aspect, the invention further comprises wiping the surface of the device with a swab or pad having a solution comprising at least one anti-infective agent and at least one polymer, prior to insertion.

The invention provides a method for reducing protein absorption and development of infections arising from insertion of a medical device through a body surface comprising coating the device with a composition comprising at least one anti-infective agent and at least one polymer. The device may be inserted through a disc comprising an antimicrobial agent or the disc may be placed around the device at the site of penetration.

### EXAMPLES

The examples listed below are illustrative and are not intended to limit the scope of the invention. The solutions were coated on insulin pump needles (MiniMed bent Needles) and dried for three minutes at about 90 degrees Celsius using a hairdryer at a distance of one-two cm from the needle surface. About 1.5 cm of the needle was coated and from about 1.0 to 1.5 cm of the needle was inserted. The needle was already connected to a delivery tube that was connected to a MiniMed 507C insulin pump. The pump used a 3 ml syringe reservoir that was filled with Humalog U-100 insulin. The insulin pump had the basal rate set at 1.2 units per hour from 4:00 am to 9:00 am, followed by 0.9 units per hour from 9:00 am to 12 noon, followed by 0.6 units per hour from noon till 4:00 am the following morning. This basal rate produced declining, fasting blood glucose levels in the mornings for a few days after the needle was first inserted into subcutaneous fatty tissue of the abdominal region.

All of the examples were used with a disc (perforated, ¼ mil thick polypropylene sheet) that was coated with the following composition: polyurethane resin (5.14 pounds (lb.)), tetrahydrofuran (11.98 lb.), methylethyl ketone (61.17 lb.), RS nitrocellulose (8.90 lb), benzalkonium chloride (1.00 lb), PCN blue/nitrocellulose paste (1.20 lb: 13.4 grams (gm) R/S PCN Blue RS N/C Paste, 5.75 gm ¼ second RS nitrocellulose, and 30.85 gm n-Butyl acetate; Penn Color 55775D).

It was observed that for uncoated needles, after two to four days, the desirable decline in fasting blood sugar levels ceases, apparently due to protein absorption around the distal portion of the needle, which is interfering with the absorption of the insulin into the surrounding tissue. All of the coatings did contain one or more agents that impart antimicrobial activity to the coated needle surface, and no infections were noted during any of the following insertion trials with the coated needles. Needles were inserted, and blood glucose levels were recorded on the order of 10-12 times per day. For Examples 1-8, the needles were removed when the fasting blood glucose levels stopped declining. It was noted that when the fasting blood glucose levels stopped declining, they would typically begin to ascend, usually rapidly, rather than exhibit a plateau behavior. After removal, the days of implantation were noted. For Examples 8-13, the needles were removed after seven days and there were no infections or decreases in fasting blood glucose levels observed for these devices.

The examples were tested by leaving the needle indwelling as long as it remained patent. The insulin pump basal rate was set so that morning-fasting blood glucose readings declined. The needle was removed when the fasting blood glucose stopped declining in the mornings. The fact that the fasting, morning blood glucose readings stopped declining was attributed to protein buildup on the needle.

For the examples listed below, the amount in grams and weight percentages are based on each component as listed including reagent solvents as applicable.

### Example 1

(733-04C1)

| | | |
|---|---|---|
| Acetonitrile | 6.01 grams | 68% |
| Denatured ethanol | 2.03 grams | 23% |
| Benzalkonium heparinate | 0.15 grams | 1.7% |
| PEG 3350 | 0.60 grams | 6.8% |

The needles with this coating composition were tested for a total of 11 insertion cycles, and resulted in an average insertion time of 4.5 days.

### Example 2

(733-19C)

| | | |
|---|---|---|
| Acetonitrile | 24.0 grams | 69% |
| Denatured ethanol | 8.01 grams | 23% |
| Irgasan (TRICLOSAN) | 0.20 grams | 0.6% |
| 2-bromo-2-nitropropane-1,3-diol | 0.10 grams | 0.3% |
| (BRONOPOL) | | |
| PEG 3350 | 2.44 grams | 7.0% |

The needles with this coating composition were tested for 14 insertion cycles, and resulted in an average insertion time of 4.4 days.

### Example 3

(733 19D)

| | | |
|---|---|---|
| Acetonitrile | 24.0 grams | 68.7% |
| Denatured ethanol | 8.01 grams | 22.9% |
| Irgasan (TRICLOSAN) | 0.20 grams | 0.6% |
| 2-bromo-2-nitropropane-1,3-diol | 0.10 grams | 0.3% |
| (BRONOPOL) | | |
| PEG 3350 | 2.44 grams | 7.0% |
| Disodium EDTA | 0.17 grams | 0.5% |

The needles with this coating composition were tested through 9 insertion cycles, and 5 resulted in an average insertion time of 5.6 days.

### Example 4

(733 47D)

| | | |
|---|---|---|
| Water | 9.00 grams | 44% |
| 2-bromo-2-nitropropane-1,3-diol | 0.07 grams | 0.3% |
| (BRONOPOL) | | |
| Polyhexamethylene biguanide (BAQUACIL) | 0.30 grams | 1.4% |
| Acrylic emulsion copolymer | 1.00 grams | 4.9% |
| 0.13% aqueous disodium EDTA | 10.0 grams | 49% |

The water and the acrylic copolymer emulsion were mixed together and added to a solution of the other three components. This order of addition produced solutions that were free of precipitate. The needles with this coating composition were tested through 12 insertion cycles, and resulted in an average insertion time of 6.1 days.

### Example 5

(733 47C)

| | | |
|---|---|---|
| Water | 9.00 grams | 44% |
| 2-bromo-2-nitropropane-1,3-diol | 0.07 grams | 0.3% |
| (BRONOPOL) | | |
| Polyhexamethylene biguanide | 0.30 grams | 1.4% |
| (BAQUACIL) | | |
| Acrylic emulsion copolymer | 1.00 grams | 4.9% |
| 0.13% aqueous disodium EDTA | 10.0 grams | 49% (0.064% EDTA) |

The disodium EDTA, water and the acrylic emulsion copolymer were mixed together first, before the 2-bromo-2-nitropropane-1,3-diol and polyhexamethylene biguanide (BAQUACIL) were added. A slight amount of precipitate was noted on the floor of the container. Therefore, the solutions were subsequently prepared using the order of addition as shown in Example 4. The needles coated with this coating composition were tested through seven insertion cycles, and resulted in an average insertion time of 5.3 days.

### Example 6

(733 81B)
Solution 1

| | |
|---|---|
| Sodium Heparin | 0.06 grams |
| 0.13% aqueous disodium EDTA | 10.0 grams |

Solution 2

| | |
|---|---|
| Acrylic emulsion copolymer | 1.00 grams |
| 0.13% aqueous disodium EDTA | 9.00 grams |

Solution 1 was added slowly to solution 2 with stirring, and then the following was added to the combined solution.

| | |
|---|---|
| 2-bromo-2-nitropropane-1,3-diol | 0.10 grams |
| (BRONOPOL) | |

This formed a stable composition that was free of precipitate. The composition had 0.3% Sodium Heparin, 50% EDTA (0.13% aqueous disodium EDTA), 5.0% Acrylic emulsion copolymer, 45% (0.13% aqueous disodium EDTA), and 0.5% 2-bromo-2-nitropropane-1,3-diol (BRONOPOL). The needles with this coating composition were tested through 22 insertion cycles, and resulted in an insertion time average of 5.9 days.

### Example 7

### (848 04B/D)

This example incorporated a basecoat and a topcoat such that the basecoat primed the needle surface, and the topcoat contained the complexing and antimicrobial agents.

Basecoat (848 04B)

| | | % base |
|---|---|---|
| Acetonitrile | 6.00 grams | 58.82% |
| Denatured ethanol | 2.00 grams | 19.61% |
| Methylethyl ketone | 0.198 grams | 1.94% |
| Nitrocellulose | 0.002 grams | 0.02% |
| PEG 8000 | 2.00 grams | 19.61% |

Total basecoat composition = 10.200 grams

Topcoat (848 32 D)

| | | |
|---|---|---|
| Water | 9.99 grams | 87.85% |
| polyhexamethylene biguanide (BAQUACIL) | 0.30 grams | 2.64% |
| 2-bromo-2-nitropropane-1,3-diol | 0.07 grams | 0.60% |
| (BRONOPOL) | | |
| Sodium EDTA | 0.013 grams | 0.11% |
| PEG 8000 | 1.0 grams | 8.80% |

Total topcoat composition = 11.373 grams

The basecoat was applied first on the needle, and dried for three minutes at ˜90 deg. C. The topcoat was applied over the base-coat and dried for three minutes at ˜90 deg. C. The needles coated with these compositions were tested through 10 insertion cycles, and resulted in an insertion time average of 6.7 days.

### Example 8

This example incorporated a basecoat (primer layer) and a topcoat such that the basecoat primed the needle surface, and the topcoat contained the complexing and antimicrobial agents.

Basecoat (848 04B)

| | | |
|---|---|---|
| Acetonitrile | 6.00 grams | 58.82% |
| Denatured ethanol | 2.00 grams | 19.61% |
| Methylethyl ketone | 0.198 grams | 1.94% |
| Nitrocellulose | 0.002 grams | 0.02% |
| PEG 8000 | 2.00 grams | 19.61% |

Total basecoat composition = 10.200 grams

Topcoat (848 32A)

| | | |
|---|---|---|
| Water | 10.00 grams | 88% |
| Polyhexamethylene biguanide (BAQUACIL) | 0.30 grams | 2.6% |
| 2-bromo-2-nitropropane-1,3-diol | 0.07 grams | 0.60% |
| (BRONOPOL) | | |
| PEG 8000 | 1.00 grams | 8.8% |

Total topcoat composition = 11.37 grams

The basecoat was applied first on the needle, and dried for three minutes at ˜90 deg. C. The topcoat was applied over the base-coat and dried for three minutes at ˜90 deg. C. The needles coated with these compositions were tested for 2 insertion cycles, and resulted in an effective insertion time average of 7.0 days, substantially longer than the 2 to 4 days patency of uncoated needles.

Table 1 summarizes the results. Each of the 8 examples had substantially longer patency than the 2-4 days of the uncoated needle controls.

TABLE I
Composition vs. Days Implanted

| COMPONENT | Ex. 1 | Ex. 2 | Ex. 3 | Ex. 4 | Ex. 5 | Ex. 6 | Ex. 7 | | Ex. 8 |
|---|---|---|---|---|---|---|---|---|---|
| (grams) | 73304C1 | 73319C | 73319D | 73347D | 73347C* | 73381B | 84804B | 84832D | 84832A |
| Acetonitrile | 6.01 | 6 | 6 | | | | 6 | | |
| ETOH | 2.03 | 2 | 2 | | | | 2 | | |
| WATER | | | | 10.5 | 9.75 | 9.750 | | 9.99 | 10 |
| MEK | | | | | | | 0.198 | | |
| Baquacil | | | | 0.30 | 0.15 | | | 0.30 | 0.30 |
| HBAK | 0.15 | | | | | | | | |
| Triclosan | | 0.05 | 0.05 | | | | | | |
| Bronopol | | 0.025 | 0.025 | 0.07 | 0.035 | 0.05 | | 0.07 | 0.07 |
| NaEDTA | | | 0.043 | 0.013 | 0.007 | 0.013 | | 0.013 | |
| Na Heparin | | | | | | 0.03 | | | |
| PEG 3350 | 0.60 | 0.61 | 0.61 | | | | | | |
| PEG 8000 | | | | | | | 2 | 1 | 1 |
| Acrylic | | | | 0.50 | 0.250 | 0.250 | | | |
| NC | | | | | | | 0.002 | | |
| # of cycles | 11 | 14 | 9 | 12 | 7 | 22 | | 10 | 2 |
| Days Implanted | 4.5 | 4.4 | 5.6 | 6.1 | 5.3 | 5.9 | N/A | 6.7 | 7.0 |

*Some precipitate was noted in this formulation. The problem was corrected in 73347D by changing the order of addition of components.

### Example 9

(Solution 848 60C)

| | | |
|---|---|---|
| Isopropyl alcohol | 3.592 grams | 59.95% |
| Water | 1.657 grams | 27.65% |
| PEG 8000 | 0.697 grams | 11.63% |
| 5-Fluorouracil | 0.0459 grams | 0.77% |

Total composition = 6.00 grams

This solution was applied twice on a needle, and dried for three minutes at 90 degrees Celsius after each application. The needle coated with this composition was tested for one insertion cycle, and was patent for 7 days, and was not infected.

### Example 10

(Solution 848-49A)

| | | |
|---|---|---|
| Ethanol | 6.00 grams | 42.58% |
| Polyhexamethylene biguanide (BAQUACIL) | 0.30 grams | 2.13% |
| Acetonitrile | 5.71 grams | 40.53% |
| PEG 8000 | 2.06 grams | 14.62% |
| 2-bromo-2-nitropropane-1,3-diol (BRONOPOL) | 0.02 grams | 0.14% |

Total composition = 14.09 grams

This solution was applied twice on a needle, and dried for three minutes at 90 degrees Celsius after each application. The needle coated with this composition was tested for one insertion cycle, and was patent for 7 days, and was not infected.

### Example 11

(Solution 848-73C)

| | | |
|---|---|---|
| Isopropyl alcohol | 6.50 grams | 53.28% |
| Water | 3.11 grams | 25.50% |
| PEG 8000 | 2.50 grams | 20.50% |
| 5-Fluorouracil | 0.08774 grams | 0.72% |

Total composition = 12.20 grams

This solution was applied twice on a needle, and dried for three minutes at 90 degrees Celsius after each application. The needle coated with this composition was tested for one insertion cycle, and was patent for 7 days, and was not infected.

### Example 12

(Solution 848-83A)

| | | |
|---|---|---|
| Ethanol | 6.01 grams | 40.47% |
| Acetonitrile | 5.75 grams | 38.72% |
| PEG 20000 | 3.00 grams | 20.20% |
| 5-Fluorouracil | 0.09002 grams | 0.61% |

Total composition = 14.85 grams

This solution was applied twice on a needle, and dried for three minutes at 90 degrees Celsius after each application. The needle coated with this composition was tested for one insertion cycle, and was patent for 7 days, and was not infected.

### Example 13

(Solution 848-83C)

| | | |
|---|---|---|
| Ethanol | 6.01 grams | 39.18% |
| Acetonitrile | 5.75 grams | 37.48% |
| PEG 20000 | 3.00 grams | 19.56% |
| MePEG-PDLLA 60:40 copolymer | 0.49 grams | 3.19% |
| 5-Fluorouracil | 0.09002 grams | 0.59% |

Total composition = 15.34 grams

This solution was applied twice on a needle, and dried for three minutes at 90 degrees Celsius after each application. The needle coated with this composition was tested for one insertion cycle, and was patent for 7 days, and was not infected.

### Example 14

Examples 14-16 show the enhanced durability of the primer/pre-coat and basecoat layers in stabilizing the topcoat layer to the device. In these examples, the topcoat was shown to remain firmly adhered to the coated device surface and retained the dye-color for more than one week when placed in an aqueous gelatin gel at room temperature. This is predictive of patency in use of one week.

This sample was prepared by first coating the stainless steel surface with primer and basecoat layers. The primer was coated on a 27-gauge stainless steel needle, and was dried at 90 degrees Celsius for three minutes. The needle was then allowed to cool at room temperature for two minutes, and was then coated over the primer with the basecoat, and dried at 90 degrees Celsius for three minutes. Next, the topcoat was applied over the other two layers, and dried for three minutes at 90 degrees Celsius, and allowed to cool for two minutes at room temperature. A final coating of topcoat was applied over the other layers, and dried for three minutes at 90 degrees Celsius, and then allowed to cool at room temperature.

The following were the compositions of the coating solutions:

Primer (58282A)

| | | |
|---|---|---|
| 5% polyethylene-co-acrylic acid polymer | 3.91 grams | 3.91% |
| Tetrahydrofuran (THF) | 74.30 grams | 74.30% |
| 37.5% w/w Epoxy resin in THF | 2.31 grams | 2.31% |
| Cyclohexanone | 15.68 grams | 15.68% |
| Polyurethane resin 25% w/w in DMA | 3.80 grams | 3.80% |

Total primer composition = 100.00 grams

Basecoat (58382A)

| | | |
|---|---|---|
| Toluene | 11.70 grams | 11.7% |
| Benzyl alcohol | 12.00 grams | 12.00% |
| Tetrahydrofuran (THF) | 37.51 grams | 37.51% |
| Cyclohexanone | 15.60 grams | 15.60% |
| Dibutylphthalate | 4.80 grams | 4.80% |
| Melamine-formaldehyde resin | 0.77 grams | 0.77% |
| (CYMEL 248-08 from CYTEC) | | |
| Butanol | 0.33 grams | 0.33% |
| Xylene | 1.93 grams | 1.93% |
| Acrylic polymer | 2.09 grams | 2.09% |
| Ethylbenzene | 0.38 grams | 0.38% |
| Nitrocellulose (70% nitrocellulose, | 10.80 grams | 10.80% |
| and 30% isopropanol) | | |
| Polyurethane resin Tecoflex SG93A | 1.99 grams | 1.99% |
| from Thermedics | | |

Total basecoat composition = 99.9 grams

Topcoat (84881B)

| | | |
|---|---|---|
| Isopropyl alcohol | 9.06 grams | 60.00% |
| Water | 4.18 grams | 27.68% |
| Polyethylene glycol 20,000 | 1.76 grams | 11.65% |
| Gentian Violet (Hucker Formula) | 0.081 grams | 0.54% |
| Tween 80 (1.0% Tween 80 solution in water). | 0.02 grams | 0.13% |

Total topcoat composition = 15.101 grams

This example was constructed to demonstrate how the use of the Primer/Basecoat combination caused the topcoat to remain firmly adhered to the coated device surface. This layer remained on the surface and retained the dye-color for more than one week when placed in an aqueous gelatin gel at room temperature.

### Example 15

This example was prepared like Example 14, but used Topcoat (84889B)

Topcoat (84889B)

| | | |
|---|---|---|
| Ethanol | 6.00 grams | 39.04% |
| Acetonitrile | 5.71 grams | 37.15% |
| MePEG/PDLLA 60/40 copolymer | 0.65 grams | 4.23% |
| Dimethylmethylene blue | trace | — |
| PEG 20,000 | 3.01 grams | 19.58% |

Total topcoat composition = 15.37 grams

This layer remained on the surface and retained the dye-color for more than one week when placed in an aqueous gelatin gel at room temperature.

### Example 16

This example was prepared like Example 14, but used Topcoat (84891C)

Topcoat (84891C)

| | | | |
|---|---|---|---|
| Isopropyl alcohol | 7.49 | grams | 55.40% |
| Water (de-ionized) | 2.96 | grams | 21.90% |
| PEG 20,000 | 3.01 | grams | 22.26% |
| Gentian Violet (Hucker Formula) | 5 | drops | — |
| 1% w/w aq. Tween 80 | 0.06 | grams | 0.44% |

Total topcoat composition = 13.52 grams

This layer remained on the surface and retained the dye-color for more than one week when placed in an aqueous gelatin gel at room temperature.

1. A medical device comprising a percutaneously insertable surface, the insertable surface comprising a surface layer comprising at least one anti-infective agent and at least one bioerodable polymer, wherein the surface layer extends the patency of the device when inserted into a patient.

2. The device of claim 1, wherein the surface layer is a coating.

3. The device of claim 2, wherein the device is coated with a composition comprising at least one anti-infective agent and at least one anti-protein absorption bioerodable polymer.

4. The device of claim 1, wherein the surface layer is deciduous.

5-8. (canceled)

9. The device of claim 1, wherein the medical device is selected from the group consisting of a needle, an infusion set or device, a peripheral venous catheter or needle, an indwelling infusion needle, a butterfly needle, a subcutaneous access device, an insulin pump needle, a patient controlled analgesia (PCA) pump needle, an arterial catheter, a central venous catheter, a dialysis catheter, a peritoneal dialysis catheter, a nephrostomy catheter, a percutaneous cystostomy catheter, an indwelling paracentesis or pleurocentesis catheter or drain, a percutaneous nephrostomy, a cystostomy tube, a spinal or epidural catheter, and a sensor.

10-13. (canceled)

14. The device of claim 1, wherein the device is a blood glucose monitor.

15. The device of claim 1, wherein the polymer is biocompatible and bioabsorbable.

16. The device of claim 1, wherein the device surface resists protein encapsulation.

17. The device of claim 1, wherein the bioerodable polymer comprises a water soluble polymer or a dispersible polymer.

18. The device of claim 1, wherein the bioerodable polymer comprises one or more polymers selected from the group consisting of polyethylene glycol, polyethylene oxide, acrylic acid or a salt or a copolymer thereof, acrylic emulsion copolymer, a polymer or copolymer of polylactic acid, a polymer or copolymer of polyglycolic acid, polyacrylamide, polyvinylpyrrolidone, polyurethane, water-soluble cellulose polymer, and methylcellulose.

19. (canceled)

20. The device of claim 1, wherein the surface layer comprises about 50% to about 99.9% bioerodable polymer.

21. The device of claim 1, wherein the surface layer comprises about 70% to about 99% bioerodable polymer.

22. The device of claim 1, wherein the bioerodable polymer is higher molecular weight polyethylene glycol (PEG).

23. The device of claim 22, wherein the polyethylene glycol (PEG) has a molecular weight of at least about 3500.

24. The device of claim 22, wherein the polyethylene glycol (PEG) has a molecular weight of at about 3500 to 35,000.

25-27. (canceled)

28. The device of claim 1, wherein the surface layer further comprises a non-bioabsorbable polymer.

29. The device of claim 28, wherein the non-bioabsorbable polymer comprises one or more polymers selected from the group consisting of acrylates, urethanes, polycarbonates, polyamides, polyesters and polyimides, styrene isobutylene, styrene polymers, cellulose esters, polystyrene, and alkylated polyvinylpyrrolidone.

30. The device of claim 1, wherein the surface layer further comprises a biostable polymer.

31. The device of claim 30, wherein the biostable polymer comprises one or more polymers selected from the group consisting of cellulose ester polymers and copolymers, polyurethanes, polyvinyl chloride, polyamides, acrylate polymers and copolymers, ethylenevinylacetate copolymers, vinylpyrrolidoneethylacetate copolymers, acetal polymers and copolymers, silicone polymers and copolymers, polyesters, polyimides and copolymers and polyetherimides.

32. The device of claim 1, wherein the surface layer comprises at least about 1 to 50% nitrocellulose.

33-41. (canceled)

42. The device of claim 1, wherein the surface layer further comprises bactericides, antibiotics, antivirals, antiseptics, antineoplastics, anticancer compounds, antifungals, anti-yeast, and/or anti-scarring agents.

43. The device of claim 1, wherein the surface layer comprises paclitaxel or rapamycin or an analog or derivative thereof.

44-45. (canceled)

46. The device of claim 1, wherein the surface layer further comprises a corticosteroid.

47-48. (canceled)

49. The device of claim 1, wherein the surface layer further comprises a non-steroidal anti-inflammatory drug (NSAID).

50. (canceled)

51. The device of claim 1, wherein the surface layer comprises two or more coating layers.

52-55. (canceled)

56. A coating composition comprising at least one anti-infective agent and at least one bioerodable polymer, wherein the coating composition, when applied to a percutaneously insertable surface of an insertable or implantable medical device, provides a surface layer that substantially extends the patency of the device when inserted into a patient.

57-74. (canceled)

75. A kit for reducing protein absorption and infection arising from insertion of a medical device through a body surface comprising:
an insertable medical device having a percutaneously insertable surface,
means for providing the insertable surface with an anti-infective, anti-protein absorption coating, wherein the coating comprises at least one anti-infective agent and at least one polymer; and
a disc comprising at least one anti-infective agent, said disc being adapted to surround and abut said percutaneously insertable surface when the device is inserted in a subject and a portion of said percutaneously insertable surface projects from an external bodily surface of the subject, and said disc is in contact with said external bodily surface of the subject.

76-82. (canceled)

83. The kit of claim 75, wherein the device is selected from the group consisting of a needle, an infusion set or device, a peripheral venous catheter or needle, an indwelling infusion needle, a butterfly needle, a subcutaneous access device, an insulin pump, a patient controlled analgesia (PCA) pump, an arterial catheter, a central venous catheter, a dialysis catheter, a peritoneal dialysis catheter, a nephrostomy catheter, a percutaneous cystostomy catheter, an indwelling paracentesis or pleurocentesis catheter or drain, a percutaneous nephrostomy, a cystostomy tube, a spinal or epidural catheter, and a sensor.

84-119. (canceled)
